SEQUENCE LISTING (14 sequences)

SEQ ID NO: 1 - DNA, 1314 bp, Solanum lycopersicum
  source 1..1314: mol_type = genomic DNA; note = Nucleotide sequence of SlBIN2 gene
atggcctcgataccgctgggacctcagcaccataatccgccggaaaatcaccatcaccaccaccttcagccgccgccgcagctcgtgcagcagcagcagcaacagcctgtgaatcgtgaactaggaggaggaggaggagctgcagtaaggaacgccggagcacgaccggaaatggaatccgaaaaggaaatgtcagctgctgttgttgagggtaatggtgccgtcactggtcacataatttccaccaccattggaggcaagaatggagaaccaaaaaggaccatcagttatatggcagagcgagttgtcggtacagggtcctttgggatagtgtttcaggcaaaatgcttggaaactggagagactgtggccataaagaaggttttacaggacaaacggtataaaaatcgtgaactacaactgatgcgcttgatggatcacccaaatgtcattactctaaagcactgcttcttttccacgactagtagagatgagctttttcttaatttggtcatggattatgtccctgaaagtttatacaaggttttaaagcactatagcaattcaaatcaaaggatgccactcatatatgtcaaactttacatgtatcagatattcagagggctggcttacattcataatgttccaaggatttgccatagagatgtgaaacctcaaaatcttttggttgatcctctgacccatcaagtcaagctgtgtgattttggaagtgcaaaagtcctggtaaatggtgaagcaaatatttcatacatttgctctcgctactacagagctccagaactcatatttggtgccacagagtatacaacatcaattgatatttggtcagcaggctgtgtccttgctgagcttcttctggggcagccgctctttcctggcgaaaatgcagtagatcaactggtggagatcatcaaggtccttggtactcctactcgggaagaaattcgatgtatgaacccaaactacacagatttcagattcccacagataaaagctcatccttggcacaaggtattccataaaagaatgcctcctgaagcaattgatcttgcctcacggcttcttcaatattcaccaagtcttcgctgtactgcactagaagcatgtgcacattcgttctttgatgagcttcgtgagcccaatgcccgtctccctaatggacgtccatttccacctcttttcaactttaaacaagagttaactggagcttcacctgatttggtcaacaagctgatccctgagcatgtgtggaggcaacttggtttgaatttcccatttcctggtgcgacgtaa

SEQ ID NO: 2 - protein, 437 aa, Solanum lycopersicum
  source 1..437: note = Amino acid sequence of protein encoded by the gene SlBIN2
MASIPLGPQHHNPPENHHHHHLQPPPQLVQQQQQQPVNRELGGGGGAAVRNAGARPEMESEKEMSAAVVEGNGAVTGHIISTTIGGKNGEPKRTISYMAERVVGTGSFGIVFQAKCLETGETVAIKKVLQDKRYKNRELQLMRLMDHPNVITLKHCFFSTTSRDELFLNLVMDYVPESLYKVLKHYSNSNQRMPLIYVKLYMYQIFRGLAYIHNVPRICHRDVKPQNLLVDPLTHQVKLCDFGSAKVLVNGEANISYICSRYYRAPELIFGATEYTTSIDIWSAGCVLAELLLGQPLFPGENAVDQLVEIIKVLGTPTREEIRCMNPNYTDFRFPQIKAHPWHKVFHKRMPPEAIDLASRLLQYSPSLRCTALEACAHSFFDELREPNARLPNGRPFPPLFNFKQELTGASPDLVNKLIPEHVWRQLGLNFPFPGAT

SEQ ID NO: 3 - RNA, 20 nt, synthetic construct
  source 1..20: mol_type = other RNA; note = sgRNA sequence
ctgggacctcagcaccataa

SEQ ID NO: 4 - DNA, 55 bp, synthetic construct
  source 1..55: mol_type = other DNA; note = DNA sequence fragments of the CRISPR/Cas9 target site of WT
atggcctcgataccgctgggacctcagcaccataatccgccggaaaatcaccatc

SEQ ID NO: 5 - DNA, 45 bp, synthetic construct
  source 1..45: mol_type = other DNA; note = DNA sequence fragments of the CRISPR/Cas9 target site of KO-5
atggcctcgataccgctgaccataatccgccggaaaatcaccatc

SEQ ID NO: 6 - DNA, 53 bp, synthetic construct
  source 1..53: mol_type = other DNA; note = DNA sequence fragments of the CRISPR/Cas9 target site of KO-10
atggcctcgataccgctgggacctcaaccataatccgccggaaaatcaccatc

SEQ ID NO: 7 - protein, 50 aa, synthetic construct
  source 1..50: note = Amino sequence fragments of the WT
MASIPLGPQHHNPPENHHHHHLQPPPQLVQQQQQQPVNRELGGGGGAAVR

SEQ ID NO: 8 - protein, 33 aa, synthetic construct
  source 1..33: note = Amino sequence fragments of bin2-5
MASIPLTIIRRKITITTTFSRRRSSCSSSSNSL

SEQ ID NO: 9 - protein, 10 aa, synthetic construct
  source 1..10: note = Amino sequence fragments of bin2-10
MASIPLASAP

SEQ ID NO: 10 - DNA, 22 bp, synthetic construct
  source 1..22: mol_type = other DNA; note = Primer SlBIN2-F
atggcctcgataccgctgggac

SEQ ID NO: 11 - DNA, 20 bp, synthetic construct
  source 1..20: mol_type = other DNA; note = Primer SlBIN2-R
ttacgtcgcaccaggaaatg

SEQ ID NO: 12 - RNA, 20 nt, synthetic construct
  source 1..20: mol_type = other RNA; note = sgRNA sequence for the target
ctgggacctcagcaccataa

SEQ ID NO: 13 - DNA, 24 bp, synthetic construct
  source 1..24: mol_type = other DNA; note = primer sequence 1 of the target
attgttatggtgctgaggtcccag

SEQ ID NO: 14 - DNA, 23 bp, synthetic construct
  source 1..23: mol_type = other DNA; note = primer sequence 2 of the target
aaacctgggactcagcaccataa

(19) United States
(12) Patent Application Publication
(10) Pub. No.: US 2024/0309393 A1
(43) Pub. Date: Sep. 19, 2024

(54) USE OF SLBIN2 GENE IN REGULATING FRUIT RIPENING AND CAROTENOID SYNTHESIS IN TOMATO

(71) Applicant: Zhejiang University, Hangzhou, Zhejiang (CN)

(72) Inventors: Qiaomei WANG, Hangzhou (CN); Fanliang MENG, Hangzhou (CN); Songshen HU, Hangzhou (CN); Lihong LIU, Hangzhou (CN); Jirong ZHENG, Hangzhou (CN); Tonglin WANG, Hangzhou (CN); Songwen LI, Hangzhou (CN); Yuanyuan LI, Hangzhou (CN)

(21) Appl. No.: 18/546,763

(22) PCT Filed: Oct. 12, 2022
(86) PCT No.: PCT/CN2022/124970

§ 371 Date: Aug. 16, 2023

(30) Foreign Application Priority Data
Dec. 11, 2021 (CN) 202111510196.5

(51) Int. Cl. C12N 15/82; C12N 9/12

(52) U.S. Cl. CPC C12N 15/825; C12N 9/1205; C12N 15/8218; C12N 15/8249

(57) ABSTRACT

The disclosure relates to the field of biotechnology, and specifically discloses the use of SlBIN2 gene in regulating fruit ripening and carotenoid synthesis in tomato. The nucleotide sequence of the SlBIN2 gene is set forth in SEQ ID NO: 1. Knockout of SlBIN2 gene promotes the accumulation of carotenoids in tomato fruits.

### CROSS-REFERENCE TO RELATED APPLICATION

This application is a national stage application of International Patent Application No. PCT/CN2022/124970, filed on Oct. 12, 2022, which claims priority of the Chinese Patent Application No. 202111510196.5, filed on Dec. 11, 2021, both of which are incorporated by references in their entities.

### REFERENCE TO SEQUENCE LISTING

A computer readable XML file entitled “HLPCTP20230302068”, created on Jun. 6, 2023, with a file size of about 17,802 bytes, contains the sequence listing for this application, has been filed with this application, and is hereby incorporated by reference in its entirety.

### TECHNICAL FIELD

The present disclosure relates to the field of biotechnology, in particular to the use of SlBIN2 gene in regulating fruit ripening and carotenoid synthesis in tomato.

### BACKGROUND

Tomato (Solanum lycopersicum) originated in South America and was introduced to China during the Wanli period of the Ming Dynasty. It is the most widely cultivated vegetable crop in China, even around the world. According to data from the Food and Agriculture Organization of the United Nations, tomato has a global yield up to 180 million tons, with a total output value of about 100 billion US dollars, ranking first among fruits and vegetables in 2019. Among them, China, with the total yield of tomato accounts for about 34.78% of the world's total yield, is the largest tomato producing country in the world.

With the improvement of national consumption level, consumers' demand for tomatoes has increased from a single demand for yield to a demand for quality. Tomato is good at sensory quality, nutritional quality and flavor quality. It is an important nutrient source beneficial to human health. It contains a variety of bioactive ingredients, such as various carotenoids like lycopene, β-carotene, lutein and so on. Carotenoids are a class of terpene compounds with unique physical and chemical properties, widely present in animals, plants, and microorganisms. In plant photosynthetic tissues and nonphotosynthetic tissues, orange pigment, yellow pigment and red pigment are produced through isoprene pathway, participate in the coloring of flowers, fruits and vegetables, and play an important role in photosynthesis and photoprotection. Meanwhile, carotenoids are essential components of human diet and precursors of vitamin A synthesis, playing an important role in human health. In addition to serving as pigments and nutrients, carotenoids are also synthetic precursors of plant hormones such as abscisic acid, strigolactones and volatile flavor compounds, involved in the growth and development, as well as the formation of flavor quality in tomatoes.

The carotenoid metabolism in tomato fruits is closely related to fruit ripening. Tomatoes, as a typical respiratory climacteric fruit, undergo significant changes in sensory properties such as color, texture, flavor, and taste during the ripening stage, as well as nutrients such as sugar, acid, vitamins, and carotenoids. Plant hormones play an important role in fruit ripening and carotenoid metabolism in tomato, and there is a network mechanism that regulates different hormones independently and interactively. Currently, it is widely believed that fruit ripening in tomato is a regulatory process dominated by ethylene and involved with multiple plant hormones.

In addition to ethylene involving in fruit ripening and carotenoid accumulation in tomato, plant hormones such as Brassinosteroid (BR) are also involved in fruit ripening and carotenoid synthesis in tomato. However, the gene function of BR signaling has not been fully verified. SlBIN2 (Solyc07g055200) is highly homologous to the Arabidopsis AtBIN2 gene and is a negative regulatory protein kinase in BR signaling, which can phosphorylate BZR1 and BES1 to negatively regulate BR signals (Vert and Chory 2006). The known functions of Arabidopsis BIN2 gene are mostly related to plant abiotic stress, cell division, cell elongation and stomatal development. At present, the known application of SlBIN2 gene is only to induce stem branching.

### SUMMARY

In view of the above technical problem, the object of the present disclosure is to provide a gene, protein involved in fruit ripening and carotenoid synthesis in tomato, and use thereof.

In order to solve the above technical problems, the disclosure provides use of SlBIN2 gene in regulating fruit ripening and carotenoid synthesis in tomato. The nucleotide sequence of SlBIN2 gene is set forth in SEQ ID NO: 1.

An improvement of the use of the disclosure is that SlBIN2 negatively regulates fruit ripening and carotenoid synthesis in tomato.

A further improvement of the use of the disclosure is that knockout of SlBIN2 gene promotes the carotenoids accumulation in tomato fruit.

In the disclosure, the protein encoded by the gene SlBIN2 has an amino acid sequence set forth in SEQ ID NO: 2.

The disclosure also provides a plasmid containing the above genes and a plant expression vector containing the above genes, the plant expression vector is an overexpression vector pGWB17-35S::SlBIN2.

The disclosure also provides host cells containing the above genes, and the host cells are cells from Escherichia coli or Agrobacterium.

The disclosure also provides a method for knocking out the SlBIN2 gene in tomato, including the following steps:
1) designing a sgRNA sequence for a target gene for editing by CRISPR/Cas9 technology, wherein the sgRNA sequence is 5′-CTGGGACCTCAGCACCATAA-3′(SEQ ID NO:3);
2) synthesizing primers according to the sgRNA sequence obtained in step 1) and constructing a CRISPR/Cas9 vector;
3) genetically transforming the vector obtained in step 2) into a plurality of wild-type tomato varieties to obtain corresponding transgenic tomato plants; then identifying plants with SlBIN2 gene knockout from the transgenic tomato plants.

The disclosure also provides the use of the above genes in construction of transgenic tomatoes, wherein the transgenic tomatoes can regulate fruit ripening and improve carotenoid content in tomato.

In the disclosure, SlBIN2-gene-overexpressing transgenic plants and gene-edited plants of tomato are constructed for the first time, and functional research is conducted. By measuring the ethylene release and carotenoid content of tomato fruit, it is found that SlBIN2 gene worked negatively in regulating ethylene release and carotenoid accumulation in tomato fruit. Through the editing of tomato SlBIN2 gene, tomato materials with early fruit ripening and higher carotenoid content are cultivated, which has a good application prospect in improving sensory and nutritional quality of tomato.

### BRIEF DESCRIPTION OF THE DRAWINGS

The embodiments of the disclosure will be explained in more detail in combination with the drawings.

FIG. 1 is a map of the vector pGWB17-35S::SlBIN2 for overexpression of SlBIN2 gene.

FIG. 2 shows the expression levels of SlBIN2 gene in SlBIN2-overexpressing lines.

FIG. 3 shows DNA sequence fragments of the CRISPR/Cas9 target site of WT (set forth in SEQ ID NO:4), KO-5 (set forth in SEQ ID NO:5) and KO-10 (set forth in SEQ ID NO:5), and sequencing results of tomato SlBIN2-gene-edited lines, such as amino sequence fragments of the WT lines (BIN2, SEQ ID NO:7) and two homozygous bin2 mutants (bin2-5, SEQ ID NO:8 and bin2-10, SEQ ID NO:9).

FIG. 4 shows the ethylene release of tomato fruits in SlBIN2-gene-overexpressing lines, gene-edited lines and wild-type lines.

FIG. 5 is a graph comparing fruit ripening process between SlBIN2-gene-overexpressing lines, gene-edited lines and wild-type tomato. Mature green (MG), Breaker (B), Pink (B+3), Red ripe (B+7). B+3 represents Pink tomato fruit (Pink, B+3), and B+7 represents Red ripe tomato fruit (Red ripe, B+7).

FIG. 6 shows the carotenoid content in tomato fruits of SlBIN2-gene-overexpressing lines, gene-edited lines and wild-type lines. There was significant difference between abcd and control (p<0.05).

### DETAILED DESCRIPTION OF THE EMBODIMENTS

The disclosure will be further described in combination with the embodiments, but the protection scope of the disclosure is not limited to these embodiments.

### 1. Acquisition of SlBIN2 Gene in Tomato and Construction of SlBIN2-Gene-Overexpressing Vector:

Primer Premier 6.0 was used to design the primers for bidirectional amplification of the gene. The leaves of wild-type tomato AC (Ailsa Craig) planted in the greenhouse of Zijingang Campus of Zhejiang University were taken, and the RNA of which was extracted. cDNA obtained by reverse transcription from the extracted RNA was used as a template (cDNA was obtained by conventional method, reference can be made to patent publication CN104561025A). The specific upstream primer was SlBIN2-F and the downstream primer was SlBIN2-R. The full-length SlBIN2 gene, namely SEQ ID No: 1, was amplified by high-fidelity enzyme PrimerSTAR.

The sequence of primers were:

SlBIN2-F:
(SEQ ID NO: 10)
5′-ATGGCCTCGATACCGCTGGGAC-3′.
SlBIN2-R:
(SEQ ID NO: 11)
5′-TTACGTCGCACCAGGAAATG-3′.

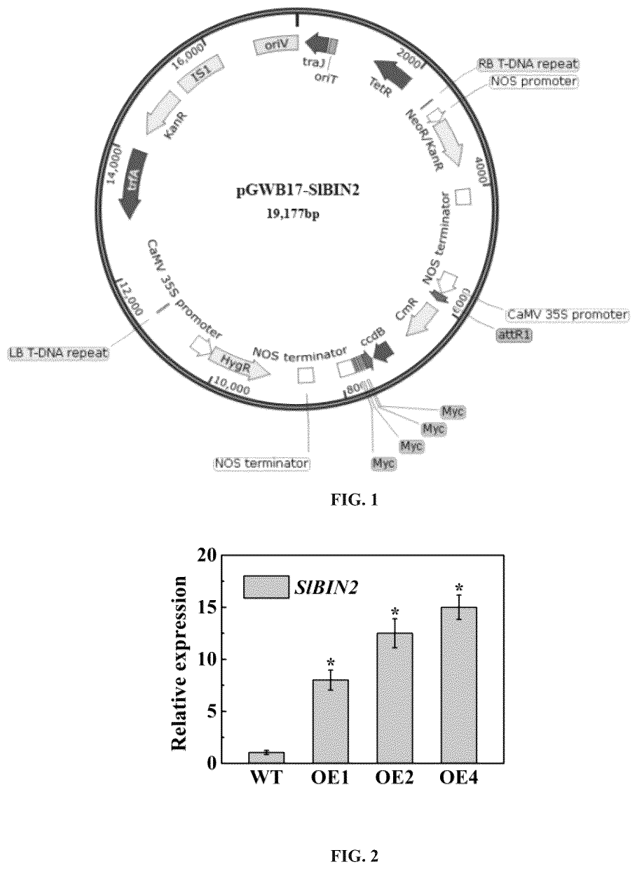

Reaction system for PCR amplification was as follows: a total reaction volume of 50 μl, including 25 μl of 2× PrimerSTAR buffer, 5 μl of dNTP Mixture, 1 μl of PrimerSTAR DNA polymerase, 14 μl of ddH₂O, 2 μl of cDNA, each 1.5 μl of upstream and downstream primers. PCR reaction procedure was as follows: pre-denaturation at 98° C. for 5 min; 35 cycles of denaturation at 98° C. for 10 s, annealing at 60° C. for 10 s, extension at 72° C. for 90 s; final extention at 72° C. for 5 min. The full length of the amplified gene was ligated to the pQB-V3 vector to obtain a recombinant plasmid. The recombinant plasmid was sent to Qingke Company for sequencing to confirm whether the sequence was correct. Then the target fragment was transferred to pGWB17 to construct an overexpression vector by homologous recombination, where the over expression vector was named pGWB17-35S::S1BIN2 (FIG. 1).

### 2. Construction of CRISPR/Cas9 Vector for Knockout of S1BIN2 Gene in Tomato

The sgRNA sequence for the target in the coding sequence of SlBIN2 gene (SEQ ID NO: 1) for CRISPR/Cas9 editing was designed using online professional software (crispr.mit.edu), where the sgRNA sequence for the target was: 5′-CTGGGACCTCAGCACCATAA-3′(SEQ ID NO:12). The corresponding primer sequences of the target, 5′-ATTGTTATGGTGCTGAGGTCCCAG-3′ (SEQ ID NO:13) and 5′-AAACCTGGGACTCAGCACCATAA-3′ (SEQ ID NO:14), were synthesized by a biotechnology company. The primers for the target were annealed and ligated into the intermediate vector AtU6-26-sgRNA-SK. The plasmids with positive PCR verification and correct sequencing were extracted and digested with Nhe I and Spe I. After electrophoresis, the fragment of about 642 bp was cut and recovered, and the fragment was recovered as sgRNA cassette. Then the sgRNA cassette was ligated into the pCAMBIA1300-pYAO: Cas9 plasmid digested by Spe I. The colony PCR identification was performed using the primer sequence on the binary vector. The identified correct monoclonal was propagated to extract the plasmid. The obtained plasmid was verified using Sal I and Kpn I digestion, and the plasmid with a length of about 670 bp was the CRISPR/Cas9 gene editing vector for SlBIN2.

### 3. Construction and Detection of Transgenic Materials:

The overexpression vector pGWB17::SlBIN2 and the CRISPR/Cas9 gene editing vector were transformed into Agrobacterium LBA4404 strain, respectively. The cotyledons of tomato were used as explants and co-cultured with the bacterial solution to obtain callus. The callus was cultured in differentiation medium and rooting medium to obtain transgenic positive seedlings. The positive transgenic plants were verified by PCR and RT-PCR.

The overexpression lines of T2 generation with separation ratio of 3:1 and high expression level (satisfied the separation ratio of 3:1 for seeds growing lateral roots and for seeds not growing lateral roots on the medium containing kanamycin (50 mg/L), and the gene expression level was increased by more than two times) were selected as the research objects (FIG. 2). In FIG. 2, WT represents wild-type tomato AC (Ailsa Craig), and OE-1, OE2 and OE4 represent the three overexpression lines of SlBIN2, respectively.

The upstream primer 5′-ATGGCCTCGATACCGCTGGGAC-3′(SEQ ID NO: 10) and downstream primer 5′-TTACGTCGCACCAGGAAATG-3′ (SEQ ID NO:11) for PCR amplification of SlBIN2 gene were synthesized. The SlBIN2 gene was amplified by 2×Taq PCR Master Mix (TIANGEN Company) using the genomic DNA of gene-edited tomato plants and their control AC as templates. The PCR amplification system was 20 μl, including 10 μl of 2×Taq PCR Master Mix, each 1 μl of upstream and downstream primers (1 μM), 1 μl of template DNA (<1 μg), 7 μl of sterile water. The PCR amplification procedure was as follows: pre-denaturation at 94° C. for 5 min; 35 cycles of denaturation at 94° C. for 30 s, annealing at 55° C. for 30 s, extension at 72° C. for 30 s; extension at 72° C. for 10 min.

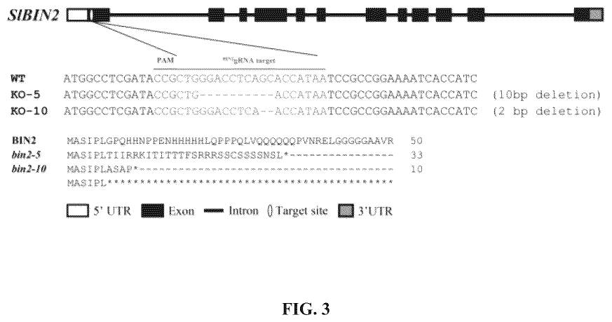

After sequencing and analysis of PCR products, two SlBIN2-gene-knocked-out lines bin2-5 and bin2-10 were identified. The coding region of the SlBIN2 gene in these two lines was deleted by 10 and 2 bases, respectively (FIG. 3), causing a frameshift mutation of the SlBIN2 gene, resulting in a loss of function of the gene. The nucleotide sequences of SlBIN2 gene in plants bin2-5 and bin2-10 lines are shown in FIG. 3.

### 4. Study on Fruit Ripening and Ethylene Release Level of Transgenic Lines

The fruits of SlBIN2-gene-overexpressing transgenic lines, gene-edited lines and wild-type tomatoes entered the mature green (MG) stage 30 days after being labeled as flowering, and entered the breaker (B) stage when the fruit top became red. Through phenotypic observation and statistics, the average number of days from the labeled flowering to the fruit breaker of wild-type tomato was about 34 days, the average number of days from the labeled flowering to the fruit breaker of the three SlBIN2-gene-overexpression lines of OE-1, OE-2 and OE-4 was about 35, 35 and 36 days, respectively, and the average number of days from the labeled flowering to fruit breaker of two SlBIN2 gene-edited lines KO-5 and KO-10 was about 32 and 33 days, respectively. The results showed that overexpression of SlBIN2 gene delayed fruit ripening in tomato, and knockout of SlBIN2 gene promoted fruit ripening in tomato.

A container for measuring ethylene release rate was made by using a 1050-ml circular fresh-keeping box (AnLiGe, China) and PM6 white straight through pneumatic diaphragm external thread joint (fssto, China). The container was punched to place the joint, and the joint was sleeved with rubber tube and the rubber tube was clamped with a flatjaw pinchcock. Each box was used as a biological replicate, and each experiment contained three biological replicates. Each box was pumped twice, each time for a technical replicate. Two to three fruits were weighed, recorded the weight and placed in one box. The flatjaw pinchcock was closed after placing the fruits in the fresh-keeping box. The box was stand for two hours under an environmental condition the same as the external treatment environment, and 1 ml of gas was extracted and injected into the gas phase.

The gas chromatograph system, including GC 6890N (Agilent Technologies, USA), air generator (KF-2L, Hangzhou KE, China) and FID detector, was adopted, where the chromatographic column was packed with Porapak Q 50/80; the carrier gas was nitrogen, the flow rate of hydrogen was 40.0 mL/mi; the flow rate of air was 40.0 mL/min; the constant tail blowing flow mode was adopted; the column temperature (heating furnace) was 100° C., the inlet temperature was 140° C. and the detector temperature was 230° C. The external standard method was used to calculate, using an ethylene standard (10-6 mol/mol, carrier gas of N₂, Nanjing Special Gas Factory, China).

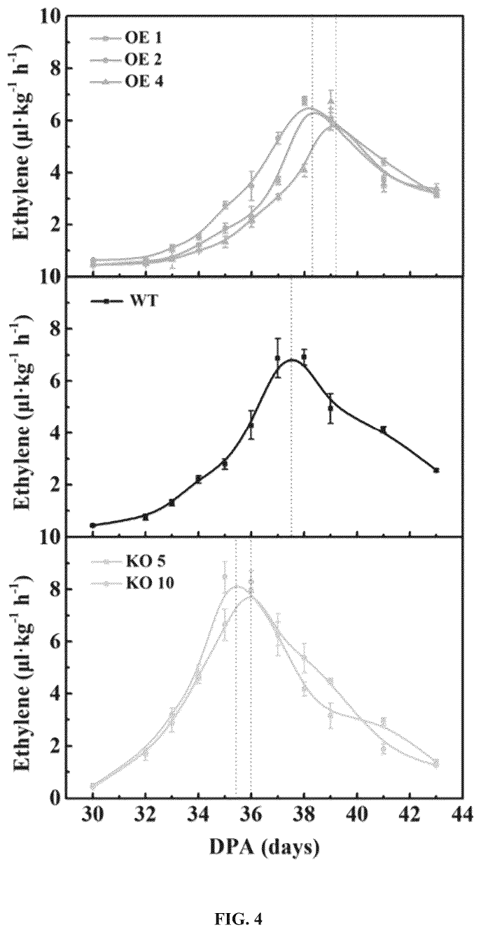

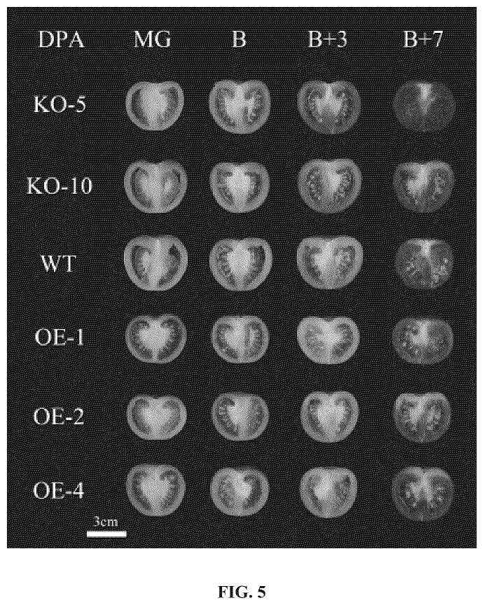

The fruits of SlBIN2-gene-overexpression transgenic lines, gene-edited lines and wild-type tomatoes entered the mature green (MG) stage 30 days after being labeled as flowering stage. The tomato fruits after being labeled as flowering stage for 30 days to 43 days were sampled to detect for the level of ethylene release. The results of ethylene release are shown in FIG. 4. According to FIG. 4, the following conclusive conclusions can be obtained: the ethylene release peaks of the two SlBIN2 gene-edited lines appeared first and the ethylene release amount was the highest, followed by the wild-type tomato fruit, and the ethylene peaks of the three SlBIN2-gene-overexpressing lines appeared latest and the release amount was the lowest. It indicated that knockout of SlBIN2 gene promoted ethylene release from tomato fruit and promoted fruit ripening in tomato, while overexpression of the SlBIN2 gene delayed ethylene release and then delayed fruit ripening in tomato.

### 5. Study on Carotenoid Content in Fruits of Transgenic Lines

After the SlBIN2-gene-overexpressing transgenic lines, gene-edited lines and wild-type tomato fruits grew to the mature green (MG) stage, the fruit samples (exocarp) were harvested. When the fruit top became red, the fruit samples of breaker stage (B) were taken. 3 days after breaker, the fruit samples of pink stage (B+3) were taken. And 7 days after breaker, the fruit samples of red ripe stage (B+7) were taken. A total of four stages of samples were taken (FIG. 5).

A certain amount of fruit tissue was grinded, and 0.5 g of fruit powder was weighed in a 50 ml centrifugal tube wrapped in foil, then 30 mL of extraction solution (volume ratio of ethane:acetone:ethanol=1:1:1) was added immediately, and shaken at 150 r/min for 30 min to obtain a mixture. Then 15 ml of double distilled water was added to the mixture and centrifuged at 1500 g for 10 min. The supernatant was filtered with 0.22 μm organic phase filter, and then concentrated to almost no liquid by nitrogen blowing apparatus. 0.5 ml of solution (volume ratio of tetrahydrofuran:acetonitrile:methanol=3:10:11) was added three times for a total of 1.5 ml. The solution in this step was used for loading.

A total of 20 μl of sample was taken and measured using Shimadzu HPLC system (Shimadzu, Kyoto, Japan), including a C18 column (5 μm particle size, 4.6 mm×250 mm, Elite analytical instruments Co., Ltd., Dalian, China), an autosampler and a SPD-M20A bipolar array detector. The mobile phase was as follows: methanol:acetonitrile=90:10 (V/V) with 0.05% triethylamine. The flow rate was 1.2 mL/min. The detection wavelength was 475 nm. The contents of lycopene, β-carotene and lutein were calculated by using an external standard method (Sigma, St Louis, MO. USA). The results were expressed as μg·g⁻¹ FW (fresh weight).

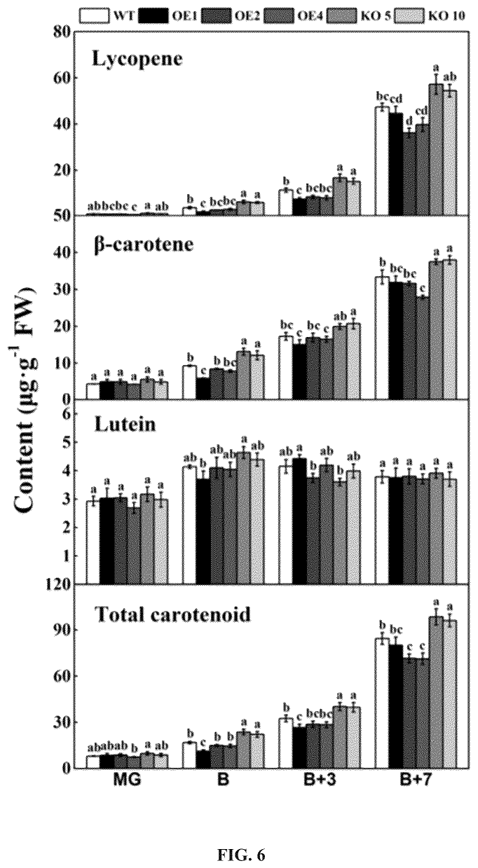

The carotenoid content of fruits of SlBIN2-gene-overexpressing lines, the gene-edited lines and wild-type tomato are shown in FIG. 6. From top to bottom, it is lycopene content, β-carotene content, lutein content and total carotenoid content. According to FIG. 6, the following conclusions can be drawn: the contents of lycopene and total carotenoids in the fruits of the two SlBIN2-gene-edited lines were significantly higher than those in the wild type lines at all stages of fruit development. At the same time, the contents of lycopene and total carotenoids in the fruits of the three SlBIN2-gene-overexpressing lines were significantly lower than those in the wild type lines at all stages of fruit development. It indicated that knockout of SlBIN2 gene promoted the accumulation of carotenoids in tomato fruit.

Finally, it should be noted that the embodiments listed above are only a number of specific embodiments of the disclosure. Obviously, the disclosure is not limited to the above embodiments and can also have many variants. All variants that those of ordinary skill in the art can directly derive or associate from the contents disclosed herein should be considered to fall within the claimed scope of the disclosure.

1. A method for regulating fruit ripening and carotenoid synthesis in tomato, comprising knocking out SlBIN2 gene, wherein the nucleotide sequence of the SlBIN2 gene is set forth in SEQ ID NO: 1, and knockout of the SlBIN2 gene promotes the accumulation of carotenoids in tomato fruit.
